(19) United States
(12) Patent Application Publication
(10) Pub. No.: US 2024/0277653 A1
(43) Pub. Date: Aug. 22, 2024

(54) USE OF FURANCARBOXYLIC ACIDS IN THE PREPARATION OF ADDITIVES FOR ANIMAL FEEDS

(71) Applicant: ANIPHA TECHNOLOGIES PTY LTD, Adelaide (AU)

(72) Inventor: Xianfeng PENG, Guangzhou (CN)

(73) Assignee: ANIPHA TECHNOLOGIES PTY LTD, Adelaide (AU)

(21) Appl. No.: 18/292,342

(22) PCT Filed: Jul. 29, 2022
(86) PCT No.: PCT/CN2022/108803

§ 371 Date: Jan. 26, 2024

(30) Foreign Application Priority Data
Jul. 30, 2021 (CN) 202110871702.7

(51) Int. Cl. A61K 31/341; A61P 3/02

(52) U.S. Cl. CPC A61K 31/341; A61P 3/02

(57) ABSTRACT

A compound of furancarboxylic acids used in the preparation of an additive for an animal feed for improving the performance of an animal is provided. The compound of furancarboxylic acids is according to Formula (I), wherein each of R₁, R₂ and R₃ is independently H or —COOH. The compound of furancarboxylic acids or a feed-acceptable salt thereof improves the performance of livestock, poultry, and aquaculture animals.

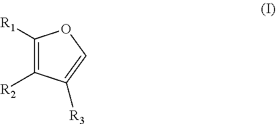

### CROSS REFERENCE TO THE RELATED APPLICATIONS

This application is the national phase entry of International Application No. PCT/CN2022/108803, filed on Jul. 29, 2022, which is based upon and claims priority to Chinese Patent Application No. 202110871702.7, filed on Jul. 30, 2021, the entire contents of which are incorporated herein by reference.

### TECHNICAL FIELD

The invention relates to the field of animal feed, in particular to the use of a compound of furancarboxylic acids, or a feed-acceptable salt thereof in the preparation of an additive for an animal feed or an animal feed for improving the performance of an animal.

### BACKGROUND

Additives for feed refer to a small or trace amount of substances added during the feed processing, production and use, including nutritional additives for feed and non-nutritional additives for feed. Non-nutritional additives for feed refer to a small or trace amount of substances incorporated into the feed to ensure or improve the quality and utilization of the feed. The non-nutritional additives for feed that are commonly used in the art to efficiently and stably improve feed utilization and improve animal performance mainly comprise high-level copper, high-level zinc, feed antibiotics, chemically synthesized antibacterials, and the like. However, the long-term use of these substances in the breeding industry has serious side effects. In order to protect the health of animals and improve the production efficiency of the breeding industry, it is an urgent problem to be solved in the field to find effective, stable and safe new additives for feed.

### SUMMARY

Accordingly, provided herein is the use of a compound of furancarboxylic acids or a feed-acceptable salt thereof in the preparation of an additive for an animal feed or an animal feed for improving the performance of an animal.

In one aspect, provided herein is the use of a compound of Formula (I), or a feed-acceptable salt thereof in the preparation of an additive for an animal feed or an animal feed for improving the performance of an animal:

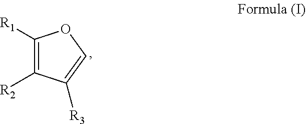

wherein each of R₁, R₂, and R₃ is independently H or —COOH.

In another aspect, provided herein is a method of improving the performance of an animal comprising administering an additive for a feed prepared by the compound according to Formula I or a feed-acceptable salt thereof, or an animal feed comprising the additive for the feed to the animal.

In another aspect, the additive for the feed prepared by the compound according to Formula I or a feed-acceptable salt thereof, or the animal feed comprising the additive for the feed provided herein is for use in improving the performance of an animal.

In another aspect, provided herein is the use of a feed composition comprising a compound of Formula (I), or a feed-acceptable salt thereof, or a combination thereof in the preparation of an additive for an animal feed for improving the performance of an animal or in the preparation of an animal feed for improving the performance of an animal.

In some embodiments, the animal feed further comprises a feedstuff for an animal feed.

In some embodiments, the animal feed further comprises an additional additive for an animal feed.

In some embodiments, the animal feed further comprises a feedstuff and additional additive for an animal feed.

In one aspect, further provided is a method of improving the performance of farmed animals.

In some embodiments, the method comprises administering a compound of furancarboxylic acids or a feed-acceptable salt thereof provided herein to a farmed animal, for example, adding a compound of furancarboxylic acids or a feed-acceptable salt thereof to the animal feed, and then feeding animals.

In some embodiments, the method comprises administering a feed composition comprising the compound of furancarboxylic acids, or a feed-acceptable salt thereof, or a combination thereof provided herein to a farmed animal.

In another aspect, the feed composition comprising the compound of furancarboxylic acids, or a feed-acceptable salt thereof, or a combination thereof provided herein is for use in improving the performance of an animal.

In some embodiments, the farmed animal is livestock, poultry, an aquaculture animal, or a pet animal at various growth stages.

In some embodiments, the farmed animal is livestock or poultry at the young stage.

In some embodiments, for the livestock, such as pig, the compound of furancarboxylic acids, or a feed-acceptable salt thereof, or a feed composition comprising the same is added in an amount of 500 to 5000 ppm, preferably 2000 ppm to the animal feed.

In some embodiments, for the poultry, such as chicken, the compound of furancarboxylic acids, or a feed-acceptable salt thereof, or a feed composition comprising the same is added in an amount of 200 to 1000 ppm, preferably 100 ppm to the animal feed.

In some embodiments, for the aquaculture animal, such as grass carp, the compound of furancarboxylic acids, or a feed-acceptable salt thereof, or a feed composition comprising the same is added in an amount of 100 to 600 ppm, preferably 500 ppm to the animal feed.

The technical effects in relation to the present invention are:

The compound of furancarboxylic acids, or a feed acceptable salt thereof, or a feed composition comprising the same provided herein is capable of improving the performance of livestock, poultry, and aquaculture animals in the animal breeding test.

Any one of embodiments of any aspect provided herein can be combined with other embodiments as long as there is no contradiction between them. In addition, in any one of embodiments of any aspect provided herein, any one of technical features can be suitable for that technical feature in other embodiments, as long as there is no contradiction between them.

The foregoing described herein merely outlines certain aspects of the present invention, but is not limited to these aspects. The above aspects and other aspects will be described in more detail below.

### DETAILED DESCRIPTION OF THE EMBODIMENTS

Certain embodiments provided herein will now be described in detail, examples of which are illustrated by the accompanying structures and formulae. The disclosure intends to encompass all alternatives, modifications and equivalents, which are all within the scope of the disclosure as defined by the appended claims. In addition, certain technical features disclosed herein, which are, for clarity, described separately in multiple independent embodiments, can also be provided in combination in an individual embodiment or in any suitable subcombination.

### Definitions and/or Terminology

“Feed-acceptable salt” as used herein, refers to a salt formed by a compound of furancarboxylic acids provided herein and an organic or inorganic base or an organic or inorganic acid that is non-toxic to animals. The term “feed-acceptable” refers to a substance or composition should be chemically or toxicologically suitable and relevant to the composition of the feed or to the farmed animal for consumption.

The “significant change” under a test condition for the study on influencing factors provided herein refers to “a change of more than 5% in the determination of the content of a test article.”

“Animal” as used herein, refers to a human or a farmed animal that is unable to convert inorganics into organics, and can only use organics as food for life activities such as ingestion, digestion, absorption, respiration, circulation, excretion, sensation, movement, and reproduction. The term “farmed animal” comprises poultry, livestock, aquaculture animals, and other animals that are legally captured in captivity, including pet animals, such as cats and dogs. The term “livestock” refers to, for example, any one of pigs, cattle, horses, goats, sheep, deer and many useful rodents. The term “poultry” intends to include, for example, chickens, ducks, geese, quails, pigeons, and the like. The term “aquaculture animal” comprises, for example, fish, shrimp, turtles, soft-shelled turtles, and the like.

“Additive for a feed” or “additive for feed” as used herein, refers to a small or trace amount of substances added during feed processing, production and use, and is classified as a nutritional additive for a feed, and a general additive for a feed which is also called a non-nutritional additive for a feed.

“Feed” as used herein, refers to a product that is processed and produced by industrialization for animal consumption. The “feed” or “animal feed” comprises a feedstuff for an animal feed and one or more additives for an animal feed, unless otherwise stated.

“Composition” as used herein, refers to a set of compounds comprising one or more of the compounds provided herein as active ingredients.

“Comprising” or “comprises” as used herein, is an open expression, which not only includes those specified herein, but does not exclude other aspects.

### Use of Furancarboxylic Acids

Provided herein is the use of a compound of furancarboxylic acids according to Formula (I), or a feed-acceptable salt thereof in the preparation of an additive for an animal feed for improving the performance of an animal:

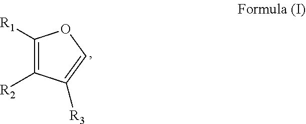

wherein each of R₁, R₂, and R₃ is independently H or —COOH.

In some embodiments, the compound of furancarboxylic acids used in the preparation of an additive for an animal feed is preferably

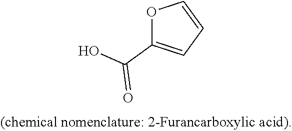

In some embodiments, the compound of furancarboxylic acids used in the preparation of an additive for an animal feed is preferably

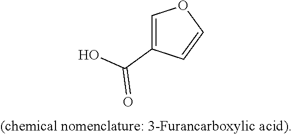

In some embodiments, the compound of furancarboxylic acids used in the preparation of an additive for an animal feed is preferably

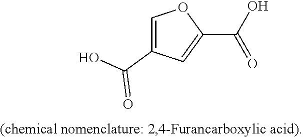 [SMILES: O=C(O)c1coc(C(=O)O)c1]

The compound of furancarboxylic acids or a feed-acceptable salt thereof is used in the preparation of a growth promoter for an animal in various growth stages, and the animal can be selected from livestock, poultry, an aquaculture animal, or a pet animal at various growth stages.

In further embodiments, the livestock include, but are not limited to, pigs, cattle, sheep, horses, rabbits, minks, or donkeys. The poultry include, but is not limited to, chickens, turkeys, ducks, geese, quails, or pigeons. The aquaculture animal includes, but is not limited to, fish, shrimp, turtles, crabs, soft-shelled turtles, bullfrogs, eels, or loaches. The pet animal includes, but is not limited to, dogs or cats of various subspecies.

In an embodiment, the compound of furancarboxylic acids or a feed-acceptable salt thereof is used in the preparation of an additive for a feed that can improve the growth performance of weaned piglets.

In another embodiment, the compound of furancarboxylic acids or a feed-acceptable salt thereof is used in the preparation of an additive for a feed that can improve the growth performance of medium pigs.

In another embodiment, the compound of furancarboxylic acids or a feed-acceptable salt thereof is used in the preparation of an additive for a feed that can improve the growth performance of chicks.

In another embodiment, the compound of furancarboxylic acids or a feed-acceptable salt thereof is used in the preparation of an additive for a feed that can improve the growth performance of fish.

### Feed Compositions

Provided herein is a feed composition comprising the compound of furancarboxylic acids, or a feed-acceptable salt thereof or a combination thereof, and a feedable excipient; wherein the feedable excipient is a feedable carrier, diluent, adjuvant, or vehicle, or a combination thereof.

“Carrier” as used herein, refers to a feedable substance capable of carrying active ingredients, improving their dispersibility, and having good chemical stability and adsorbability, including organic or inorganic carriers. The organic carrier is a material containing a lot of crude fiber, including but not limited to, corn flour, corncob flour, wheat bran, rice husk flour, defatted rice bran, rice mill by-product, corn stalk flour, peanut husk flour, and the like. The inorganic carrier is a mineral, which is mainly divided into calcium salts and silicon oxides and is used for preparing a trace element premix, including but not limited to calcium carbonate, silicate, vermiculite, zeolite, sepiolite, and the like.

“Diluent” as used herein, refers to a substance that distributes the starting additive evenly in the material, and dilutes the high-concentration additive into a low-concentration premix, which can separate trace components from each other and reduce the interaction between active ingredients to increase the stability of active ingredients without affecting the physicochemical properties of related substances, including organic or inorganic diluents. Organic diluents include, but are not limited to, corn flour, degerminated corn flour, dextrose (glucose), sucrose, semolina with bran, sautéed soybean meal, wheat middlings, corn gluten meal, and the like; and inorganic diluents include, but are not limited to, limestone, calcium dihydrogen phosphate, shell powder, kaolin (white clay), sodium chloride, and sodium sulfate.

“Adjuvant” as used herein, refers to a wetting agent that induces the inherent viscosity of the substance, a binder that binds the substance together, a disintegrant that breaks the entire sheet of the substance into many fine particles, a retention aid that reduces friction between particles, or an anti-sticking agent that prevents material sticking, including but not limited to magnesium stearate, talc, vegetable oil, magnesium lauryl sulfate, starch, starch slurry, water, inorganic salts, dextrin, powdered sugar, and the like.

“Vehicle” as used herein, refers to a solvent required to dissolve or disperse solids, including but not limited to, water, ethanol, glycerin, and the like.

In some embodiments, the feed composition further comprises an additional additive for an animal feed and/or a feedstuff for an animal feed.

The additive for the animal feed is a nutritional additive for a feed, a general additive for a feed, or a pharmaceutical additive for a feed.

The nutritional additive for the feed refers to a small or trace amount of substances added to a formula feed to balance feed nutrients, improve feed utilization, and directly exert nutritional effects on animals, including amino acids, amino acid salts and their analogs, vitamins and vitamin-like substances, mineral elements and their complexes (chelates), microbial enzyme preparations, or non-protein nitrogen.

The general additive for the feed is also called a non-nutritional additive, and refers to some non-nutritional substances added to the feed to improve feed utilization, ensure feed quality and quality, and be beneficial to animal health or metabolism, including growth promoters, insect repellent health care agents, flavoring and food attractants, feed conditioners, feed preparations, feed storage agents, and Chinese herbal medicine additives.

In some embodiments, the composition comprising a compound of furancarboxylic acids or a feed-acceptable salt thereof further comprises one or more of a nutritional additive for a feed, a non-nutritional additive for a feed, and a pharmaceutical additive for a feed.

In some embodiments, the feedstuff for the animal feed comprises feedable substances, including grains and their processed products, oilseeds and their processed products, legume seeds and their processed products, tubers, roots and their processed products, other seeds, fruit products and their processed products, forage grass, roughage and its processed products, other plants, algae and their processed products, dairy products and their by-products, terrestrial animal products and their by-products, fish, other aquatic organisms and their by-products, minerals, microbial fermentation products and by-products, other feedstuffs.

### Use of Feed Compositions

Provided herein is the use of a feed composition comprising the compound of furancarboxylic acids, or a feed-acceptable salt thereof.

In some embodiments, the feed composition comprising the compound of furancarboxylic acids or a feed-acceptable salt thereof is used in the preparation of an additive for an animal feed.

In some embodiments, the additive for the animal feed prepared by using the feed composition comprising the compound of furancarboxylic acids or a feed-acceptable salt thereof is an additive for a feed for livestock, poultry, aquaculture animals, or pet animals.

In specific embodiments, the feed composition comprising the compound of furancarboxylic acids or a feed-acceptable salt thereof is used in the preparation of an additive for a feed for livestock, wherein the livestock include, but are not limited to, pigs, cattle, sheep, horses, rabbits, minks and the like at various growth stages.

In specific embodiments, the feed composition comprising the compound of furancarboxylic acids or a feed-acceptable salt thereof is used in the preparation of an additive for a feed for poultry, wherein the poultry include, but is not limited to, chickens, ducks, geese, pigeons and the like at various growth stages.

In specific embodiments, the feed composition comprising the compound of furancarboxylic acids or a feed-acceptable salt thereof is used in the preparation of an additive for a feed for aquaculture animals, wherein the aquaculture animals include, but are not limited to, fish, shrimp, crabs, soft-shelled turtles, eels and the like at various growth stages.

In specific embodiments, the feed composition comprising the compound of furancarboxylic acids or a feed-acceptable salt thereof is used in the preparation of an additive for a feed for pet animals, wherein the pet animals include, but are not limited to, cats and dogs in captivity.

In some embodiments, the additive for the animal feed prepared from the composition comprising the compound of furancarboxylic acids or a feed-acceptable salt thereof is a premix, a multi-premix, aqueous solution, or a granule.

In some embodiments, the feed composition comprising the compound of furancarboxylic acids or a feed-acceptable salt thereof is used in the preparation of an animal feed.

In some embodiments, the animal feed prepared by using the feed composition comprising the compound of furancarboxylic acids or a feed-acceptable salt thereof is a feed for livestock, poultry, aquaculture animals, or pet animals.

In specific embodiments, the feed composition comprising the compound of furancarboxylic acids or a feed-acceptable salt thereof is used in the preparation of a feed for livestock, wherein the livestock include, but are not limited to, pigs, cattle, sheep, horses, rabbits, minks and the like at various growth stages.

In specific embodiments, the feed composition comprising the compound of furancarboxylic acids or a feed-acceptable salt thereof is used in the preparation of a feed for poultry, wherein the poultry include, but is not limited to, chickens, ducks, geese, pigeons and the like at various growth stages.

In specific embodiments, the feed composition comprising the compound of furancarboxylic acids or a feed-acceptable salt thereof is used in the preparation of a feed for aquaculture animals, wherein the aquaculture animals include, but are not limited to, fish, shrimp, crabs, soft-shelled turtles, eels and the like at various growth stages.

In specific embodiments, the feed composition comprising the compound of furancarboxylic acids or a feed-acceptable salt thereof is used in the preparation of a feed for pet animals, wherein the pet animals include, but is not limited to, cats and dogs in captivity.

In some embodiments, the feed prepared from the feed composition comprising the compound of furancarboxylic acids or a feed-acceptable salt thereof is a single feed, a concentrated feed, a formula feed, a multi-premix, or a concentrate supplement.

In specific embodiments, the formula feed is a complete formula feed.

### Methods for Improving the Performance of Farmed Animals

In some feeding embodiments, the additive for the feed comprising the compound of furancarboxylic acids or a feed-acceptable salt thereof is administered to an animal along with the feed, which can significantly improve the performance of the animal.

In some embodiments, the additive for the feed is a premix, a multi-premix, a granule, or aqueous solution, which is mixed with the animal feed and consumed by animals.

The animals are livestock, poultry, aquaculture animals, or pet animals.

In specific embodiments, the livestock include, but are not limited to, pigs, cattle, sheep, horses, rabbits, minks and the like at various growth stages. The poultry include, but is not limited to, chickens, ducks, geese, pigeons and the like at various growth stages. The aquaculture animals include, but are not limited to, fish, shrimp, crabs, soft-shelled turtles, eels and the like at various growth stages. The pet animals include, but are not limited to, cats and dogs in captivity.

In an embodiment, the additive for the feed comprising the compound of furancarboxylic acids or a feed-acceptable salt thereof is administered to a weaned piglet along with the feed, which can significantly improve average daily gain and feed conversion in weaned pigs.

In an embodiment, the additive for the feed comprising the compound of furancarboxylic acids or a feed-acceptable salt thereof is administered to a broiler along with the feed, which significantly reduces the feed conversion ratio in broilers and improves the feed conversion.

In an embodiment, the additive for the feed comprising the compound of furancarboxylic acids or a feed-acceptable salt thereof is administered to fish along with the feed.

In an embodiment, the additive for the feed comprising the compound of furancarboxylic acids or a feed-acceptable salt thereof is administered to a puppy along with the feed.

In another feeding embodiment, the feed composition comprising the compound of furancarboxylic acids or a feed-acceptable salt thereof is administered to an animal, which can significantly improve the performance of the animal.

Optionally, the feed composition is a premix of an additive for a feed, a multi-premix of an additive for a feed, a granule, or aqueous solution, which is administered to an animal along with the feed.

In an embodiment, the feed composition is a premix of an additive for a feed.

In an embodiment, the feed composition is a composite premix of an additive for a feed.

Optionally, the feed composition is a concentrated feed, a formula feed, a multi-premix, or a concentrate supplement, which is directly consumed by animals as an animal feed.

In an embodiment, the feed composition is a complete formula feed.

Certain embodiments provided herein will now be described in detail, examples of which are illustrated by the accompanying structures and formulae. The disclosure intends to encompass all alternatives, modifications and equivalents, which are within the scope of the disclosure as defined by the appended claims. In addition, certain technical features disclosed herein, which are, for clarity, described separately in multiple independent embodiments, can also be provided in combination in an individual embodiment or in any suitable subcombination.

### Example 1 Effects of Furancarboxylic Acids on Performance of Weaned Piglets

A total of 120 65-day-old Duroc-landrace-yorkshire swine with similar body weight were randomly divided into 4 groups, each with 3 replicates, half male and female, 10 pigs in each group. The pigpen and utensils were disinfected before the test. During the period of test, pigs were kept in separate pens under the same feeding and management conditions. During the test, the test pigs were fed with food and water ad libitum, and were fed twice a day. The test groups are shown in Table 1, among which, group I was the control group, and was only fed with the basal diet; test groups II, III and IV were fed with the basal diet comprising 2000 ppm of a compound of furancarboxylic acids shown in Table 1, respectively. Each test group did not add other antioxidant components and growth promoters in the whole feeding process. The test lasted 28 days, and the performances of the test pigs, for example, the average daily feed intake (ADFI, g/d*pig), average daily weight gain (ADG, g/d*pig) and feed conversion ratio (FCR) of each pig were counted. The test results are expressed as “mean”, as shown in Table 1. The results showed that the feed intake of the test pigs in the test groups fed with the basal diet comprising a compound of furancarboxylic acids was not significantly affected, but the average daily gain was significantly increased while the feed conversion ratio decreased by about 5.6%, compared with the control group.

TABLE 1
Effects of furancarboxylic acids on performance of pigs

| | | Number of test | | | |
|---|---|---|---|---|---|
| | Test articles | pigs | ADFI | ADG | FCR |
| group I | — | 10*3 | 1187 | 450 | 2.637 |
| group II | 2-Furancarboxylic acid | 10*3 | 1274 | 512 | 2.489 |
| group III | 3-Furancarboxylic acid | 10*3 | 1240 | 498 | 2.491 |
| group IV | 2,4-Furandicarboxylic | 10*3 | 1263 | 507 | 2.490 |
| | acid | | | | |

### Example 2 Study on the Use of Furancarboxylic Acids in a Feed for Chicken

This experiment adopted a single-factor randomization design. 480 1-day-old broilers with an average body weight of 50 g were selected and randomly divided into 4 groups, with 6 replicates in each group, half male and female, 20 yellow-feathered broilers per replicate. The chicken house and utensils were disinfected before the test. During the period of test, broilers were kept in cages under the same feeding and management conditions. The basal diet is mainly based on corn-soybean meal, and other antioxidants and growth promoters are not added during the whole feeding. The test groups are shown in Table 2, among which, group I was the control group, and was only fed with the basal diet; test groups II, III and IV were fed with the basal diet comprising 1000 ppm of a compound of furancarboxylic acids shown in Table 2, respectively. The test lasted 20 days, and the test broilers were fed with food and water ad libitum, and were fed twice a day. Test broilers in each replicate were weighed at 21 days of age (stop feeding for 12 hours and keep water). The feed consumption of the test broilers was counted, and the average daily feed intake (ADFI, g/d*broiler), average daily weight gain (ADG, g/d*broiler) and feed conversion ratio (FCR) of each test broiler were calculated.

The test results are expressed as “mean”, as shown in Table 2. The results showed that the effect of the feed intake of the test chickens in each test group was not significant, but the average daily gain of the test chickens was significantly increased and the feed conversion ratio was significantly decreased, compared with the control group.

TABLE 2
Effects of furancarboxylic acids on performance of broilers

| | | Number of test | | | |
|---|---|---|---|---|---|
| | Test articles | broilers | ADFI | ADG | FCR |
| group I | — | 20*6 | 32.6 | 14.8 | 2.20 |
| group II | 2-Furancarboxylic acid | 20*6 | 35.2 | 16.9 | 2.08 |
| group III | 3-Furancarboxylic acid | 20*6 | 34.0 | 16.4 | 2.07 |
| group IV | 2,4-Furandicarboxylic | 20*6 | 36.5 | 17.4 | 2.10 |
| | acid | | | | |

### Example 3 Study on the Use of Furancarboxylic Acids in a Feed for Fish

### (1) Experimental Material

Test fish: The test fish used were the healthy and lively grass carp fingerlings of the same year and were reared in a large cage (4×2×1.5 m³) for 4 weeks before being used for formal breeding tests. The experimental system was a small floating cage (with size of 1.1×1.1×1.1 m³); each small cage is equipped with a bubbling head, and bubbling 24 hours a day. The small cages and the temporary cages were placed in a test facility, a 3500 m² pond, the pond depth was about 1.5 m, and the pond water was fully aerated bottom water. During the test, 240 grass carps that had been starved for 1 day were randomly divided into 3 groups, each group was provided with 4 replicates, and 20 fish were placed in each replicate. Each replicate was weighed and placed into 12 cages, respectively fed with a test feed supplemented with a compound of furancarboxylic acids.

Test feed: The test feed was prepared according to the formula in Table 3, and 500 ppm of a compound of furancarboxylic acids were added to different test groups according to Table 4. The feedstuff used was ultrafinely grinded and then passed through the Jiangsu Muyang extruder to make a floating extruded feed with a particle size of 3 mm. The mold release temperature was 130° C., and 3% soybean oil was sprayed externally through the oil injection equipment, and sealed in a cool place for use.

TABLE 3
Feed formula for grass carp and chemical ingredients (wt %)

| Ingredients | Content (%) | Ingredients | Content (%) |
|---|---|---|---|
| fish meal | 9.0 | soybean oil | 3.0 |
| hog casing | 3.0 | phospholipid | 9.0 |
| powder | | rapeseed meal | |
| soybean meal | 12.0 | gluten | 4.0 |
| rapeseed meal | 12.0 | blood cell powder | 2.0 |
| MSG protein | 3.0 | Vc-Phosphate | 0.1 |
| wheat middlings | 12.6 | calcium dihydrogen | 1.8 |
| | | phosphate | |
| flour | 17.0 | choline chloride | 0.2 |
| bentonite | 0.70 | multivitamins | 0.1 |
| rice bran | 10.0 | micro-mineral | 0.5 |
| | | premix | |

TABLE 4
Growth-promoting test groups of furancarboxylic acids

| Groups | Test articles | Amount (ppm) |
|---|---|---|
| control | — | — |
| group I | 2-Furancarboxylic acid | 500 |
| group II | 3-Furancarboxylic acid | 500 |

### (2) Experimental Method

Procedures: The experiment adopts artificial food restriction feeding, the feeding amount is adjusted once a week, the feeding level (based on the initial body weight) of each group is exactly the same, feeding twice a day (at 7:30 am and 15:00 pm), and the total feeding amount is 580 g per replicate. The test lasted 8 weeks. The water quality was monitored regularly during the test. The water temperature was 26.88±3.08° C., with DO>5.0 mg O L⁻¹, pH 7.8, ammonia nitrogen <0.50 mg N L⁻¹, and nitrite nitrogen <0.05 mg N L⁻¹ during the whole breeding process.

Parameter statistics: During the test, the fish in each cage were weighed as a whole 1 day of stopping feeding, and their weight gain (WG, %), feed conversion rate (FCR) and survival rate (SR, %) were calculated as follows:

Weight gain (WG, %)=100×(average final weight−average initial weight)/average initial weight;

Feed conversion rate (FCR)=feed intake/weight gain of fish;

Survival rate (SR, %)=100×number of fish at the end of the experiment/number of fish at the beginning of the experiment.

### (3) Experimental Results

The results of the effect of furancarboxylic acids on the performance of grass carps are shown in Table 5. The results showed that the test groups added with a compound of furancarboxylic acids were better than the control group in terms of weight gain and feed conversion rate, and had obvious growth-promoting effects. In addition, the survival rate and anti-hypoxia ability of grass carp were significantly improved in test groups.

TABLE 5
Test results of the use of furancarboxylic
acids in a feed for aquaculture animals

| | Average initial | Average final | Weight | | |
|---|---|---|---|---|---|
| | weight (g) | weight (g) | gain (%) | (FCR) | SR (%) |
| control | 251.25 | 574.64 | 133.15 | 1.56 | 55.00 |
| group I | 249.81 | 586.87 | 134.96 | 1.48 | 82.50 |
| group II | 246.56 | 589.33 | 138.98 | 1.48 | 83.75 |

The foregoing examples merely represent several embodiments of the invention, and there are other embodiments to perform the invention. Correspondingly, the embodiments of the invention will be illustrated as examples, but should not be regarded as limiting the scope of the present invention, and may also be modifications made within the scope of the present invention and within the same inventive concept or be equivalents added in the claims.

What is claimed is:

1. A method of preparing of an additive for an animal feed for improving the performance of an animal comprising adding a compound of Formula (I), or a feed-acceptable salt thereof, or a feed composition comprising the compound or salt to the additive for the animal feed, 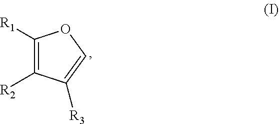
wherein each of R₁, R₂, and R₃ is independently H or —COOH.

2. The method according to claim 1, wherein each of R₁ and R₃ is H, and R₂ is —COOH; or R₁ is —COOH, and each of R₂ and R₃ is H; or each of R₁ and R₃ is —COOH, and R₂ is H.

3. The method according to claim 1, wherein the additive for the animal feed is an additive for a feed for livestock, poultry, aquaculture animals, or pet animals at various growth stages.

4. The method according to claim 1, wherein the additive for the animal feed is a growth promoter.

5. A method of preparing of an animal feed for improving the performance of an animal comprising adding a compound of Formula (I), or a feed-acceptable salt thereof, or a feed composition comprising the compound or salt to the animal feed, 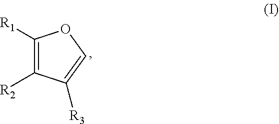
wherein each of R₁, R₂, and R₃ is independently H or —COOH.

6. The method according to claim 5, wherein each of R₁ and R₃ is H, and R₂ is —COOH; or R₁ is —COOH, and each of R₂ and R₃ is H; or each of R₁ and R₃ is —COOH, and R₂ is H.

7. The method according to claim 5, wherein the animal feed is a feed for livestock, poultry, aquaculture animals, or pet animals at various growth stages.

8. The method according to claim 5, wherein the animal feed further comprises a feedstuff for an animal feed.

9. The method according to claim 5, wherein the animal feed further comprises an additional additive for an animal feed.

10. The method according to claim 9, wherein the additional additive for the animal feed is a nutritional additive for a feed, a non-nutritional additive for a feed, or a pharmaceutical additive for a feed.

11. The method according to claim 2, wherein the additive for the animal feed is an additive for a feed for livestock, poultry, aquaculture animals, or pet animals at various growth stages.

12. The method according to claim 2, wherein the additive for the animal feed is a growth promoter.

13. The method according to claim 6, wherein the animal feed is a feed for livestock, poultry, aquaculture animals, or pet animals at various growth stages.
